(19) United States
(12) Patent Application Publication
(10) Pub. No.: US 2019/0380837 A1
(43) Pub. Date: Dec. 19, 2019

(54) FEMORAL IMPLANT SYSTEMS WITH A PLURALITY OF MODULAR TROCHLEA COMPONENTS

(71) Applicant: Charles Riviere Hip & Knee Replacement Limited, London (GB)

(72) Inventor: Charles RIVIERE, London (GB)

(73) Assignee: Charles Riviere Hip & Knee Replacement Limited, London (GB)

(21) Appl. No.: 16/446,185

(22) Filed: Jun. 19, 2019

Related U.S. Application Data
(60) Provisional application No. 62/687,030, filed on Jun. 19, 2018.

(51) Int. Cl. A61F 2/38

(52) U.S. Cl. CPC A61F 2/3859; A61F 2/3836; A61F 2002/30329; A61F 2002/3863; A61F 2/3877

(57) ABSTRACT

Knee implants, prosthesis systems, prosthesis kits and related surgical methods including a femoral implant system is provided. The femoral implant system comprises at least one condylar component comprising medial condyle articular surface portion, a lateral condyle articular surface portion, and an intercondylar notch between the medial and lateral condyle articular surface portions. The femoral implant system also comprises a plurality of modular trochlea components each comprising an articular surface with a differing trochlea groove. The at least one condylar component and the plurality of modular trochlea components are separate components. One condylar component and one modular trochlea component are configured to be implanted into a distal end portion of a femur and cooperate to form replacement articulation surfaces of the femur that substantially replicate a native articular surface portion of the distal end portion of the femur.

### CROSS REFERENCE TO RELATED APPLICATIONS

This application perfects and claims priority benefit of U.S. Provisional Patent Application No. 62/687,030, filed Jun. 19, 2018, and entitled Femoral Implants with a Modular Trochlea Component, which is hereby incorporated herein by reference in its entirety

### TECHNICAL FIELD

The present disclosure generally relates to implants, systems, kits and surgical methods related knee replacement prosthesis. More specifically, the present disclosure relates to knee implants, knee prosthesis systems, knee prosthesis kits, and surgical methods including a femoral implant with a modular trochlea component.

### BACKGROUND

Joint arthroplasty is a well-known surgical procedure by which a diseased and/or damaged natural joint is replaced by a prosthetic joint. The most widely-used type of knee prosthesis for implantation into a patient during a total knee arthroplasty (TKA) procedure includes three components: a femoral component (typically metallic) that attaches to the distal femur a tibial component (or tray) that attaches to the proximal tibia (typically metallic); and an insert (also called a bearing or an inlay) (typically polymeric, such as UHMWPE) that fits between the femoral and tibial components. A knee prosthesis may be a total knee prosthesis designed to replace the femoral-tibial interface of both condyles of the patient's femur and the patella-femoral joint or a partial knee replacement designed to replace the femoral-tibial interface of a single condyle of the patient's femur (a uni-compartmental or uni-condylar replacement) and/or a patellofemoral replacement. A typical total orthopedic knee prosthesis aims to create a straight limb and align the extensor mechanism (quadriceps muscle-patella-patella tendon-anterior tibial tuberosity).

Knee prostheses of varying mobility are used. For example, a knee prosthesis may include a “fixed” tibial bearing insert, in which the insert does not move relative to the tibial component. Alternatively, a knee prosthesis may include a “mobile” tibial bearing insert, in which the insert rotates upon a smooth platform of the tibial component. Some mobile bearing implants may provide an enhanced bearing surface between a liner and a femoral component in order to reduce liner wear without increasing risk of tibia component loosening.

Knee prostheses may include a patella component that is secured to the patient's natural patella such that its posterior surface articulates with the femoral component during extension and/or flexion of the knee. Some types of patella components include a dome-shaped polymer bearing and a conforming or anatomic bearing that is designed to conform with the bearing surfaces of the femoral component.

The type of orthopedic knee prosthesis used to replace a patient's natural knee may also depend on whether the patient's posterior cruciate ligament is retained or sacrificed (i.e., removed) during surgery. For example, if the patient's posterior cruciate ligament is damaged, diseased, and/or otherwise removed during surgery, a posterior stabilized knee prosthesis may be used to provide additional support and/or control at later degrees of flexion. Alternatively, if the posterior cruciate ligament is intact, a cruciate retaining knee prosthesis may be used.

While certain aspects of conventional technologies have been discussed to facilitate disclosure of Applicant's inventions, the Applicant in no way disclaims these technical aspects, and it is contemplated that their inventions may encompass one or more conventional technical aspects.

In this specification, where a document, act or item of knowledge is referred to or discussed, this reference or discussion is not an admission that the document, act or item of knowledge or any combination thereof was, at the priority date, publicly available, known to the public, part of common general knowledge, or otherwise constitutes prior art under the applicable statutory provisions; or is known to be relevant to an attempt to solve any problem with which this specification is concerned.

### SUMMARY

Briefly, the present disclosure satisfies the need for knee implants, knee prosthesis systems, knee prosthesis kits, and surgical methods for total knee arthroplasty (TKA) that include a femoral implant with a modular trochlea component. In various embodiments, a plurality of modular femoral implants or components are disclosed. The modular femoral implants may be configured to facilitate recreation of a patient's native trochlea anatomy. The modular nature of the femoral implants allows for a kinematic alignment technique that facilitates reproduction of the individual anatomy (e.g., restore the patient's native trochlea anatomy).

The femoral implants include a condylar component and a modular trochlea component. Systems and/or kits according to the present disclosure may include a plurality of femoral implants with modular trochlea components that are selected for implantation based, at least in part, on the best fit with the patient's native anatomy (e.g., to reproduce the individual patient's trochlea anatomy when performing a TKA). Such systems and/or kits may include modular trochlea components with various frontal groove alignments (e.g., neutral, varus or valgus) relative to the distal condylar line. The differing modular trochlea components may likewise join or couple to the condylar components. Such systems and/or kits may also include modular trochlea components of differing sizes (e.g., thicknesses). The femoral implants (e.g., the condylar components thereof) may be configured as cruciate retaining (PCL or PCL & ACL cruciate ligaments) implants, postero-stabilized implants, or medial pivot implants.

In one aspect, the present disclosure provides a femoral implant for a knee replacement prosthesis comprising a condylar component and a trochlea component. The condylar component comprises a medial condyle portion defining a medial condyle articular surface portion, a lateral condyle portion defining a lateral condyle articular surface portion, and an intercondylar notch formed between the medial and lateral condyles. The trochlea component comprises an articular surface that defines a trochlea groove. The condylar component and the trochlea component are distinct components. The condylar component and the trochlea component are coupled together and configured to be implanted into the distal end portion of a femur to form articulation surfaces thereof.

In some embodiments, the orientation of the trochlea groove of the trochlea component substantially corresponds to the native trochlea groove of the femur. In some such embodiments, the trochlea groove of the trochlea component is a neutrally oriented trochlea groove that is angled less than or equal to about 1 degree from an axis that is normal to a distal femoral joint line defined by the medial and lateral condyles. In some other such embodiments, the trochlea groove of the trochlea component is a varus oriented trochlea groove that is angled greater than about 1 degree in the medial direction from an axis that is normal to a distal femoral joint line defined by the medial and lateral condyles. In some other such embodiments, the trochlea groove of the trochlea component is a valgus oriented trochlea groove that is angled greater than about 1 degree in the lateral direction from an axis that is normal to a distal femoral joint line defined by the medial and lateral condyles.

In some embodiments, the articular surface of the trochlea component comprises a convex medial articular surface portion on a medial side of the trochlea groove and a convex lateral articular surface portion on a lateral side of the trochlea groove. In some such embodiments, the trochlea groove, medial articular surface portion and the lateral articular surface portion of the articular surface of the trochlea component are each defined by at least one radius, and wherein at least one of a radius of the trochlea groove, a radius of the medial articular surface portion and a radius of the lateral articular surface portion substantially corresponds to that of a corresponding portion of the native trochlea articular surface anatomy of the distal end portion of the femur. In some other such embodiments, the maximum thickness of the trochlea component is configured such that at least one of the medial articular surface portion, the lateral articular surface portion and the trochlea groove is positioned at substantially the same location as that of a corresponding portion of the native trochlea articular surface of the femur.

In another aspect, the present disclosure provides a prosthesis system comprising at least one condylar component and a plurality of modular trochlea components. The at least one condylar component comprises a medial condyle portion defining a medial condyle articular surface portion, a lateral condyle portion defining a lateral condyle articular surface portion, and an intercondylar notch formed between the medial and lateral condyles. The plurality of modular trochlea components each comprise an articular surface that defines a trochlea groove. The at least one condylar component and the plurality of modular trochlea components are separate components. One condylar component and one modular trochlea component cooperate to replace a distal end of a femur.

In some embodiments, the plurality of modular trochlea components comprises modular trochlea components with differing orientations of the trochlea groove thereof. In some such embodiments, the plurality of modular trochlea components comprises at least one modular trochlea component with a neutrally oriented trochlea groove that is angled less than or equal to about 1 degree from an axis that is normal to a distal femoral joint line defined by the medial and lateral condyles of the one condylar component. In some other such embodiments, the plurality of modular trochlea components comprises at least one modular trochlea component with a varus oriented trochlea groove that is angled greater than about 1 degree in the medial direction from an axis that is normal to a distal femoral joint line defined by the medial and lateral condyles of the one condylar component. In some other such embodiments, the plurality of modular trochlea components comprises at least one modular trochlea component with a valgus oriented trochlea groove that is angled greater than about 1 degree in the lateral direction from an axis that is normal to a distal femoral joint line defined by the medial and lateral condyles of the one condylar component. In some other such embodiments, the plurality of modular trochlea components comprises at least one modular trochlea component that includes a trochlea groove that substantially corresponds to the orientation of the native trochlea groove of the distal end of the femur.

In some embodiments, the articular surface of each of the plurality of modular trochlea components define a convex medial articular surface portion on a medial side of the trochlea groove and a convex lateral articular surface portion on a lateral side of the trochlea groove. In some such embodiments, the trochlea groove, medial articular surface portion and the lateral articular surface portion of the articular surface of each of the plurality of modular trochlea components are each defined by at least one radius, and the plurality of modular trochlea components comprises modular trochlea components with differing radiuses of at least one of the trochlea groove, the medial articular surface portion and the lateral articular surface portion. In some other such embodiments, the plurality of modular trochlea components comprises modular trochlea components with differing maximum thicknesses of the trochlea component at at least one of the medial articular surface portion, the lateral articular surface portion and the trochlea groove.

In another aspect, the present disclosure provides a kit comprising at least one condylar component, a plurality of modular trochlea components and a guide. The at least one condylar component comprises a medial condyle portion defining a medial condyle articular surface portion, a lateral condyle portion defining a lateral condyle articular surface portion, an intercondylar notch formed between the medial and lateral condyles, and an engagement surface configured to engage a distal femur comprising at least one peg. The plurality of modular trochlea components each comprises an articular surface that defines a trochlea groove and an engagement surface configured to engage the distal femur comprising at least one peg. The guide comprises a plurality of apertures and is configured to facilitate the formation of cavities within the distal femur via the plurality of apertures for mating with pegs of one of the condylar and trochlea components. One condylar component and one modular trochlea component cooperate to replace a distal end of a femur.

In some embodiments, the guide is a drill guide configured to facilitate the formation of the cavities within the distal femur via a drill passing through the plurality of apertures. In some embodiments, the medial condyle portion defines a medial portion of the engagement surface of the at least one condylar component and the lateral condyle portion defines a lateral portion of the engagement surface of the at least one condylar component, and each of the medial and lateral portions of the engagement surface of the at least one condylar component comprises a peg. In some embodiments, the engagement surface of each of the modular trochlea components comprises a medially positioned first peg, a laterally positioned second peg, and a third peg that is positioned proximally or distally and at least partially between the first and second pegs along the medial-lateral direction.

The knee implants, knee prosthesis systems and/or kits and surgical methods of the present disclosure may address one or more of the problems and deficiencies of the art discussed above. For example, the traditional total knee replacement prostheses, including femoral implants therefore,

However, it is contemplated that the knee implants, knee prosthesis systems, knee prosthesis kits, and surgical methods of the present disclosure may prove useful in addressing other problems and deficiencies in a number of technical areas. Therefore, the disclosed and claimed inventions should not necessarily be construed as limited to addressing any of the particular problems or deficiencies discussed herein.

Certain embodiments of the presently-disclosed knee implants, knee prosthesis systems, knee prosthesis kits, and surgical methods have several features, no single one of which is solely responsible for their desirable attributes. Without limiting the scope of the knee implants, knee prosthesis systems, knee prosthesis kits and surgical methods of the present disclosure (e.g., those that are defined by the claims that follow), their more prominent features will now be discussed briefly. After considering this discussion, and particularly after reading the section of this specification entitled “Detailed Description,” one will understand how the features of the various embodiments disclosed herein provide a number of advantages over the current state of the art.

These and other features and advantages of this disclosure will become apparent from the following detailed description of the various aspects of the disclosure taken in conjunction with the appended claims and the accompanying drawings.

### BRIEF DESCRIPTION OF THE DRAWINGS

The present disclosure will hereinafter be described in conjunction with the following drawing figures, which are not necessarily drawn to scale for ease of understanding, wherein the same reference numerals retain their designation and meaning for the same or like elements throughout the various drawings, and wherein:

FIG. 1A is a perspective posterior view of a femoral implant including a condylar component and a modular trochlea component according to the present disclosure;

FIG. 1B is a perspective exploded view of the femoral implant of FIG. 1;

FIG. 2 is an anterior view of a femoral implant including a condylar component and a modular trochlea component with a neutral frontal groove alignment according to the present disclosure;

FIG. 3 is an anterior view of a femoral implant including a condylar component and a modular trochlea component with a varus frontal groove alignment according to the present disclosure;

FIG. 4 is an anterior view of a femoral implant including a condylar component and a modular trochlea component with a valgus frontal groove alignment according to the present disclosure;

FIG. 5 illustrates a side view of a prior art femoral implant implanted into a distal femur illustrating understuffing of a patella-femoral joint;

FIG. 6 is an anterior view of a femoral implant according to the present disclosure illustrating the radiuses/heights of portions of modular trochlea components with respect to a patellar flexion axis as indicated in FIG. 5;

FIG. 7 depicts a side view of modular trochlea components according to the present disclosure with differing thicknesses;

FIG. 8 is a perspective view of a distal femur being prepared to receive a femoral implant with a condylar component and a modular trochlea component according to the present disclosure;

FIG. 9 is a perspective view of a modular femoral implant implanted onto the of distal femur of FIG. 8;

FIG. 10 is a cross-sectional view of the femoral implant of FIG. 9 illustrating the junction between the condylar and the trochlear modular components;

FIGS. 11A-11F illustrate differing views of a postero-stabilized femoral implant including a condylar component and a modular trochlea component according to the present disclosure;

FIGS. 12A-12F illustrate differing views of a first cruciate and/or bi-cruciate retaining femoral implant including a condylar component and a modular trochlea component according to the present disclosure; and

FIGS. 13A-13F illustrate differing views of a second cruciate and/or bi-cruciate retaining femoral implant including a condylar component and a modular trochlea component according to the present disclosure.

### DETAILED DESCRIPTION

Aspects of the present disclosure and certain features, advantages, and details thereof, are explained more fully below with reference to the non-limiting embodiments illustrated in the accompanying drawings. Descriptions of well-known materials, fabrication tools, processing techniques, etc., are omitted so as to not unnecessarily obscure the inventions in detail. It should be understood, however, that the detailed description and the specific example(s), while indicating embodiments of inventions, are given by way of illustration only, and are not by way of limitation. Various substitutions, modifications, additions and/or arrangements within the spirit and/or scope of the underlying inventive concepts will be apparent to those skilled in the art from this disclosure.

In this detailed description and the following claims, the words proximal, distal, anterior, posterior, medial, lateral, superior, inferior and anatomical references are used in reference to both the orthopaedic implants described herein and a patient's natural anatomy. Such terms have well-understood meanings in both the study of anatomy and the field of orthopaedics. Use of such anatomical reference terms in the specification and claims is intended to be consistent with their well-understood meanings unless noted otherwise. For example, “proximal” means the portion of an implant nearest the torso, while “distal” indicates the portion of the implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Any examples of parameters are not exclusive of other parameters of the disclosed embodiments. Components, aspects, features, configurations, arrangements, uses and the like described, illustrated or otherwise disclosed herein with respect to any particular embodiment may similarly be applied to any other embodiment disclosed herein.

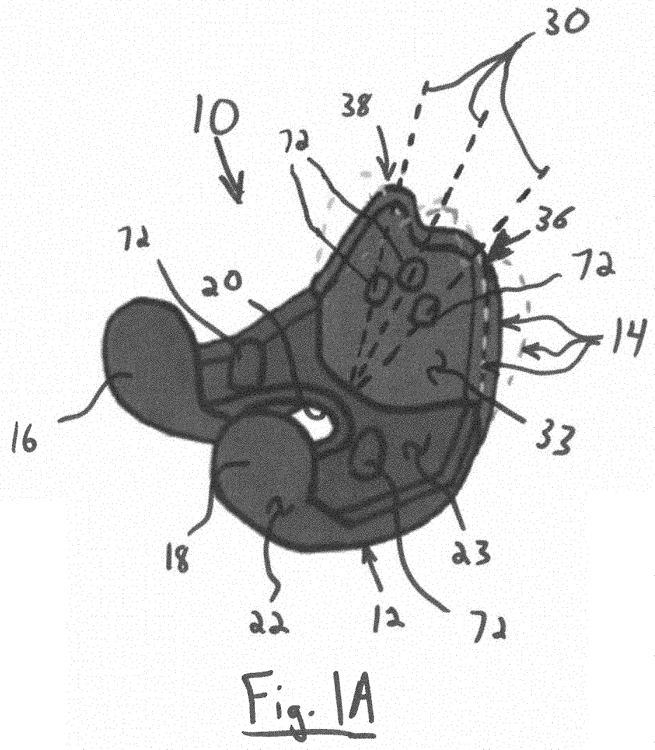

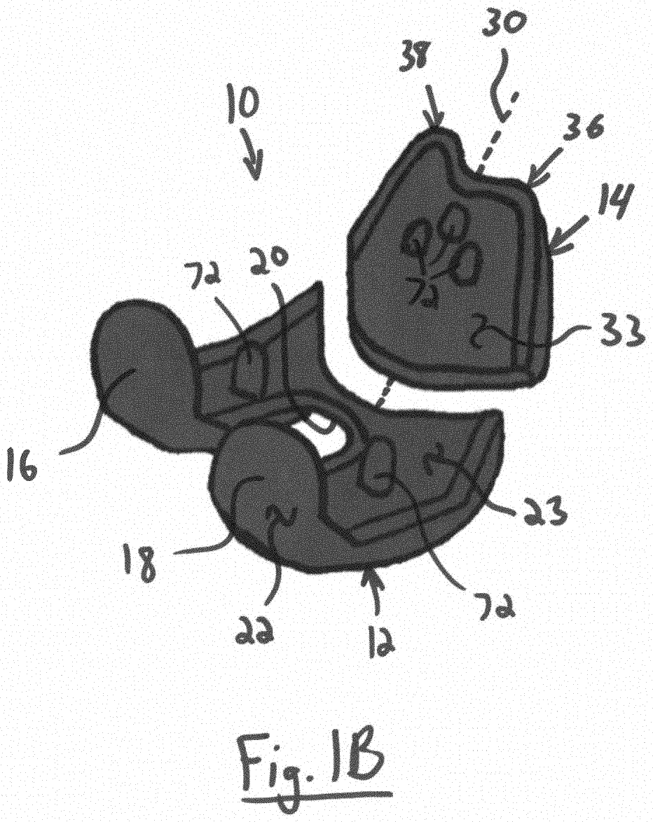

Referring to the drawings, and with particular reference to FIGS. 1A-4, there is illustrated exemplary femoral knee implants or prostheses 10 (e.g., endoprostheses) that include a condylar or femoral component, portion or implant 12 and a modular trochlea (or trochlear) component, portion or implant 14 for a total knee arthroplasty or replacement. The condylar component 12 and the trochlea component 14 may be separate and distinct elements or parts (each comprised of one or more elements or parts). The femoral implants 10 may also comprise knee prosthesis systems, kits and surgical methods for repairing and/or replacing a knee joint. The modular trochlea component 14 allows for selective reproduction (at least partially) of an individual patient's natural, native or constitutional trochlea anatomy via a specifically selected or determined trochlea component 14 when performing a knee replacement.

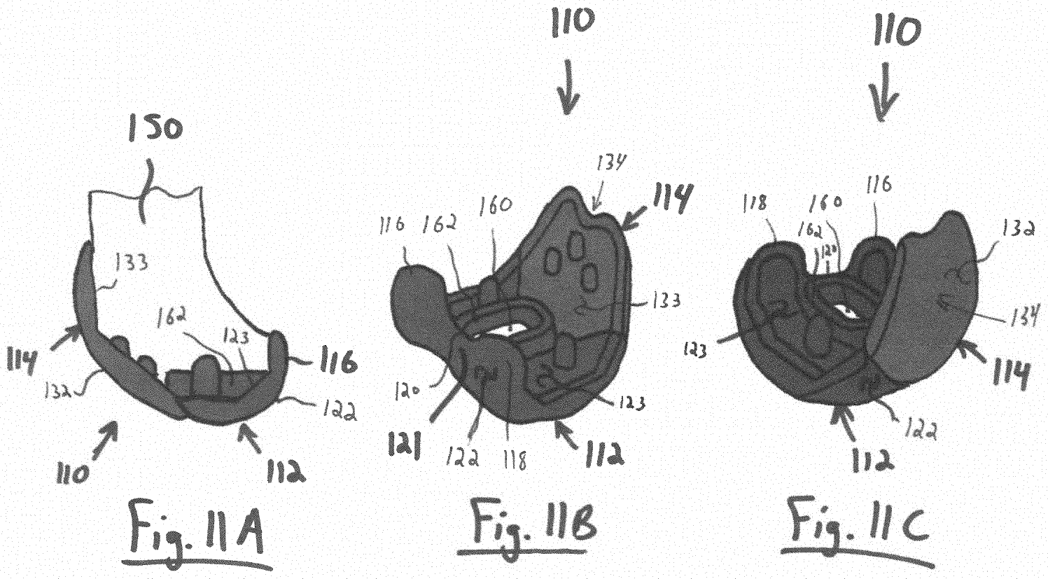

As shown in FIGS. 1A-4, the condylar component 12 and the trochlea component 14 are configured to cooperate with each other to form the femoral knee implant 10. In a cooperating arrangement, the condylar component 12 and trochlea component 14 are configured to be coupled to a surgically-prepared surface of the distal end of a patient's femur to form at least portion of the distal aspect of the femur, as shown in FIGS. 8-10. The condylar component 12 may form the distal and posterior portion of the knee implant 10, and the trochlea component 14 may form the anterior (and potentially proximate) portion of the femoral knee implant 10, as shown in FIGS. 1A-4.

As shown in FIGS. 1A-4, the condylar component 12 of the femoral implant 10 may be configured to emulate the configuration of a patient's natural femoral condyles, and, as such, includes a medial condyle portion 16 and a lateral condyle portion 18. The medial and lateral condyle portions 16, 18 are configured (e.g., curved) in a manner which mimics the condyles of a natural femur. The medial and lateral condyle portions 16, 18 are spaced apart from one another (e.g., generally along the medial-lateral direction) thereby defining an intercondylar notch 20 therebetween, as shown in FIGS. 1A-4. In some embodiments, the medial and lateral condyle portions 16, 18 are substantially identical or symmetrical with each other. In other embodiments, the medial and lateral condyle portions 16, 18 are asymmetrical (e.g., the lateral condyle 18 being defined by a smaller or larger sagittal radius than that of the medial condyle 16). In some embodiments, the medial and lateral condyle portions 16, 18 may diverge from each other in the medal-lateral plane.

It should be appreciated that the illustrative orthopaedic knee prostheses, and component or aspects thereof, described and illustrated herein are configured to replace a patient's right knee. However, in other embodiments, the knee implants disclosed herein may be configured to replace a patient's left knee. In such embodiments, it should be appreciated that the aspects of the prostheses should be rearranged or reconfigured in a mirrored or inverse relationship (e.g., at least along the medial-lateral direction) to properly accommodate a patient's left knee. Regardless, the features and concepts described herein may be incorporated in a knee prosthesis configured to replace either knee joint of a patient.

As also shown in FIGS. 1A-4, the condylar component 12 defines an outer articular surface 22 configured to engage and articulate with one or more bearing surfaces of a natural tibia and/or a tibial bearing implant (not shown), for example. The articular surface 22 includes a medial condyle articular surface portion defined by the medial condyle 16 and a lateral condyle articular surface portion defined by the lateral condyle 18. In some embodiments, the articular surface 22 of the condylar component 12 may also include an intermediate articular surface portion (e.g., intercondylar fossa portion) extending between the medial and lateral condyle articular surface portions, as shown in FIGS. 1A-4. The medial and lateral condyle articular surface portions may be convex surface portions (e.g., convexly curved surface portions) and the intermediate articular surface portion may be a concave surface portion (e.g., a concavely curved surface portion) of the articular surface 22.

The intermediate articular surface portion may form at least a portion of the intercondylar notch 20. The intermediate articular surface portion of the articular surface 22 of the condylar component 12 may or may not form a portion of a trochlea groove (and/or sulcus line) of the implant 10, as described further below. For example, the intermediate articular surface portion may or may not engage and articulate with a patella (e.g., a natural patella and/or a patella implant) during movement of the knee joint.

The medial and lateral condyle articular surface portions may be shaped to emulate the configuration of the patent's natural femoral condyles, and, as such, are configured (e.g., curved) in a manner that mimics the articular surfaces of the condyles of a natural femur. In use, the medial and lateral condyle articular surface portions of the condylar component 12 articulate on at least one corresponding bearing surface during extension and/or flexion of the patient's knee. In some embodiments, the medial and lateral condyle articular surface portions of the articular surface 22 are spherical, such as at least along the medial-lateral and sagittal planes.

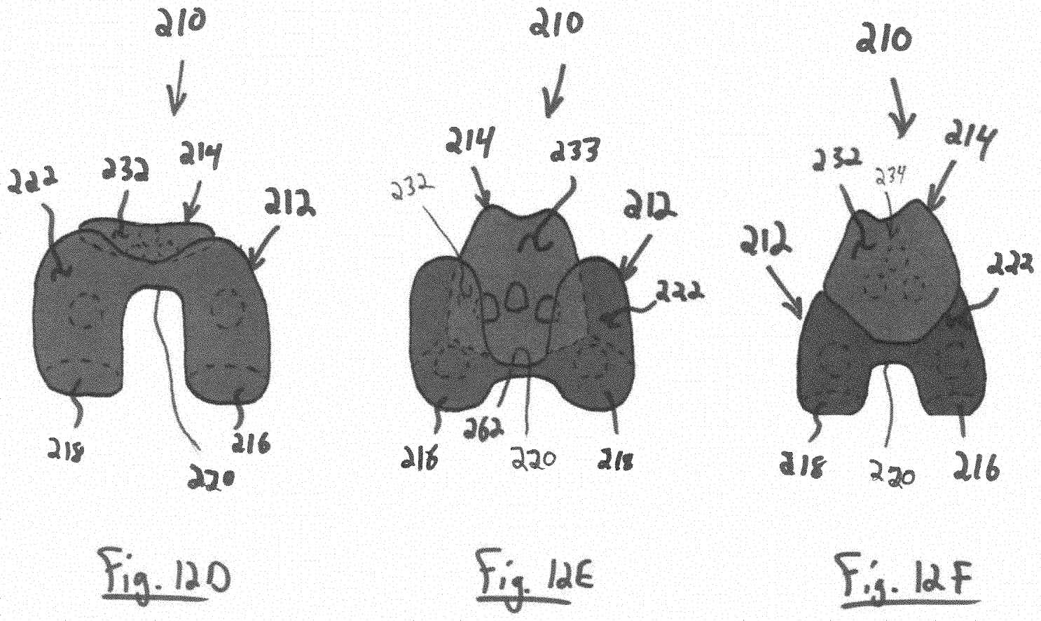

The illustrative orthopaedic femoral knee prostheses 10 of FIGS. 1A-4, 6 and 9 is embodied as a posterior cruciate-retaining knee prosthesis. That is, the condylar component 12 of the femoral knee implant 10 is embodied as a posterior cruciate-retaining femoral component. It should be appreciated that in other embodiments the orthopaedic femoral knee prosthesis 10 may be a posterior cruciate-sacrificing knee prosthesis, such as a posterior stabilized knee prosthesis, as described further below with respect to FIGS. 11A-13F.

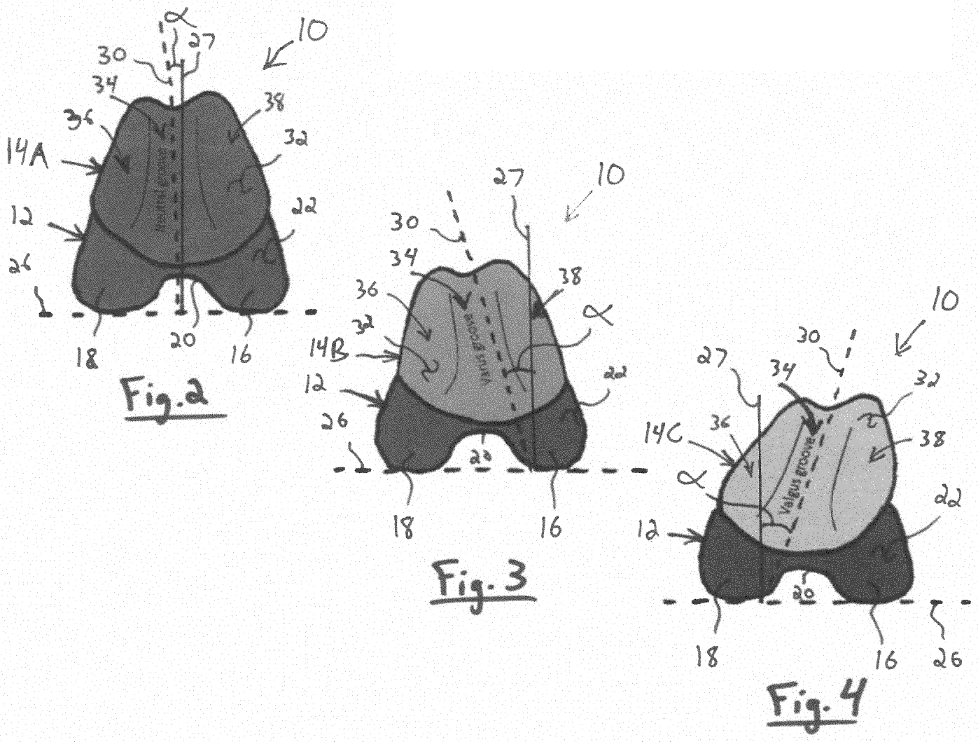

As shown in FIGS. 1A-5, 10A and 10, the trochlea component 14 of the knee implant 10 may be configured to cooperate with the condyle component 12 when implanted on the distal portion of a femur. The trochlea component 14 also defines an outer articular surface 32 configured to engage and articulate with one or more surfaces, such as a patient's patella (e.g., a natural patella and/or a patella implant), during extension and/or flexion of the patient's knee (not shown). The articular surface 32 of the trochlea component 12 and the articular surface 22 of the condyle component 12 may cooperate to form a substantially continuous or uniform outer articulation surface of the femoral implant 10, as shown in FIGS. 2-4, 9 and 10A-12 (e.g., extending from a joint therebetween).

As shown in FIGS. 2-4 and 7, the trochlea component 14 may include a medial facet portion 36 at a medial portion or side, a lateral facet portion 38 at a lateral portion or side, and a trochlear groove portion 34 positioned between the medial and lateral facet portions 36, 38 (along the medial-lateral direction). The medial and lateral facet portions 36, 38 may be convex surface portions (e.g., convexly curved/arcuate portions) and the trochlear groove portion 34 may be a (medially-laterally) concave surface portion (e.g., a concavely curved/arcuate portion) extending medially-laterally between the surface portions of the medial and lateral facet portions 36, 38. The medial and lateral facet portions 36, 38 may thereby form convex (medially-laterally) medial and lateral articular surface portions (e.g., a medially-laterally convexly curved/arcuate surface portions) of the articular surface 32. The trochlear groove portion 34 may thereby form a concave (medially-laterally) articular surface portion (e.g., a medially-laterally concavely curved/arcuate surface portion) of the articular surface 32. The medial and lateral facet portions 36, 38 may extend further in the anterior direction than the trochlear groove 34. The trochlear groove 34 may thereby comprise an indentation or depression in the articular surface 32, or otherwise positioned below the medial and lateral facet portions 36, 38 in a thickness direction of the trochlea component 14.

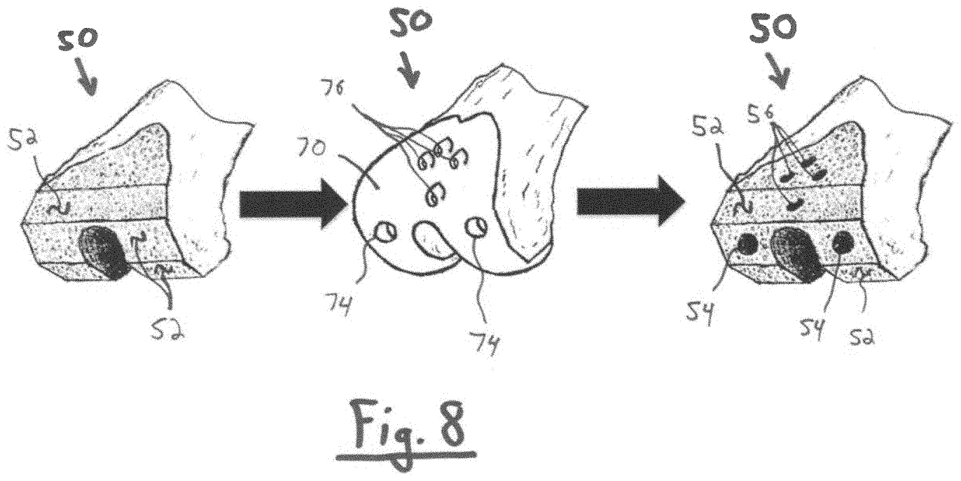

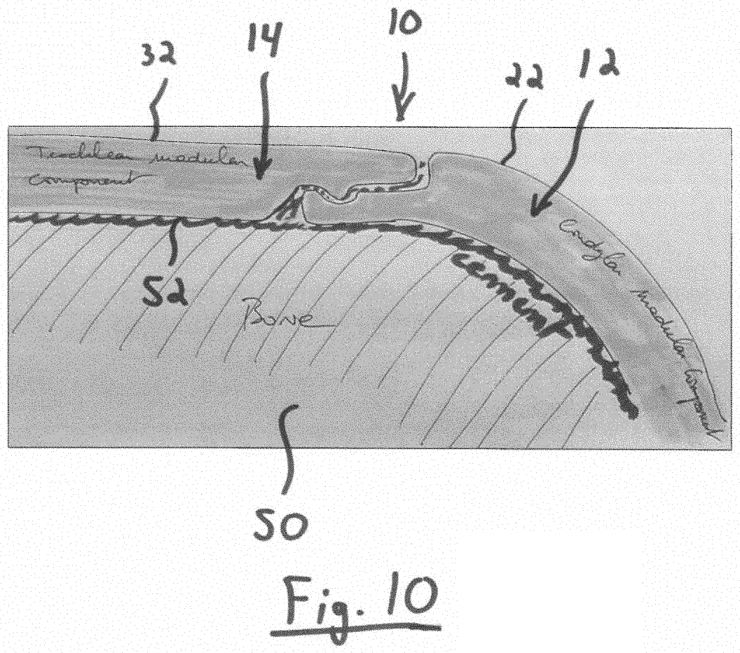

In some embodiments, the trochlear groove portion 34 (and thereby the articular surface portion thereof) may be convex (e.g., a convexly curved/arcuate portion) along or in a proximal-distal direction (e.g., the proximal-distal direction and the medial-lateral direction being perpendicular), as shown in FIGS. 5 and 7. Similarly, in some embodiments, the medial and lateral facet portions 36, 38 (and thereby the articular surface portions thereof) may be convex (e.g., convexly curved/arcuate portions) along or in the proximal-distal direction.

The trochlear groove 34 of the trochlea component 14 may be configured to receive a patient's patella (natural and/or artificial). In some embodiments, the femoral implant 10 may include a patella component (not shown) that is configured to be received in the trochlear groove 34 and articulate with the articular surface 32 of the trochlea component 14 (and potentially a portion of the articular surface 22 of the condylar component 12) during extension and flexion of the patient's knee. It should be appreciated that in other embodiments the patient's natural patella may be received in the trochlear groove 34 and articulate with the articular surface 32 of the trochlea component 14 (and potentially a portion of the articular surface 22 of the condylar component 12) during use.

As shown in FIGS. 2-4, the trochlear groove 34 of the trochlea component 14 may define or include a longitudinal axis 30. For example, the longitudinal axis 30 may approximate or generalize the path of the trochlear groove 34, as shown in FIGS. 2-4. In some embodiments, the longitudinal axis 30 may extend along the center and/or bottom (or deepest) portion of the trochlea component 14. In this way, the center and/or bottom/deepest portion of the trochlea component 14 may define the longitudinal axis 30.

As also shown in FIGS. 2-4, the medial condyle portion 16 of the condylar component 12 has a peak or distal-most point on the articular surface 22 thereof. Similarly, lateral condyle portion 18 of the condylar component 12 has a peak or distal-most point or apex on the articular surface 22 thereof, such as in the sagittal plane. As shown in FIGS. 2-4, the apexes of the medial and lateral condyle portions 16, 18 define a distal femoral joint line/axis or distal transverse plane 26. In some embodiments, when the condylar component 12 is implanted, the distal femoral joint line 26 may be substantially aligned with the native distal femoral joint line of the native femoral condyles (i.e., aligned with a line/axis joining the distal points of the medial and lateral native femoral condyles). The implanted condylar component 12 may thereby be in kinematic alignment. As also shown in FIGS. 2-4, a reference axis, line or plane 27 may extend orthogonal to the line/plane 26. The reference line 27 may extend vertically or along the sagittal plane. In some embodiments, the reference line 27 may correspond, or extend substantially parallel to, the mechanical axis of the patient's leg. In some embodiments, the reference line 27 may extend parallel to the patient's inferior-superior axis (not shown). As explained further below, the reference line 27 may be utilized as a reference to the orientation of the trochlear groove 34 of the trochlea component 14.

As shown in FIGS. 2-4, a trochlear angle α of the trochlear groove 34 of the trochlea component 14 may be defined between the longitudinal axis 30 and the orthogonal reference line or plane 27 (which is perpendicular or normal to the distal femoral joint line or plane 26). As also shown in FIGS. 2-4, the angle of trochlear groove 34 of the trochlea components 14 may vary between differing trochlea components 14. One of the plurality of differing modular trochlea components 14 may be used with the same condylar component 12 (or one or more of a plurality of differing condylar components 12) to form a femoral implant 10 best suited for the particular patient. In this way, a femoral implant 10 may include a specifically-selected trochlea component 14 (from a plurality of differing trochlea components 14) with a trochlear groove 34 orientate at a trochlear angle α that substantially or most closely mimics or recreates the natural or native patient's trochlea anatomy, for example. The Applicant has determined that when current monoblock femoral knee implants (i.e., single structure femoral implants without interchangeable parts) are kinematically aligned, surgeons are likely to reproduce the native (or constitutional) patient-specific distal femoral joint line orientation in frontal and axial planes, but fail to reproduce the highly inter-individually variable trochlea anatomy (groove orientation and trochlea stuffing and/or spacing). The Applicant has also determined that such non-restoration of the patient-specific trochlea anatomy might affect patella kinematics and result in patella-femoral related complications (e.g., patella implant accelerated wear and loosening, patella instability, patella fracture, anterior knee pain, reduced knee flexion, reduce function, reduced satisfaction and the like) affecting a patient's clinical and/or functional outcome (such as, but not limited to, range of motion and gait). The Applicant has further determined that full, or even partial, restoration of a patent's patient-specific trochlea anatomy can be clinically beneficial.

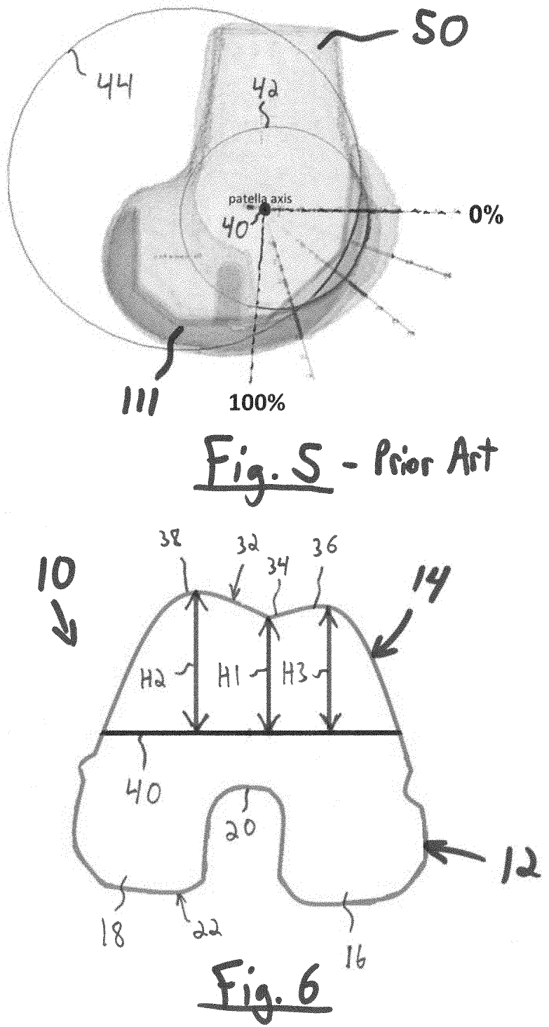

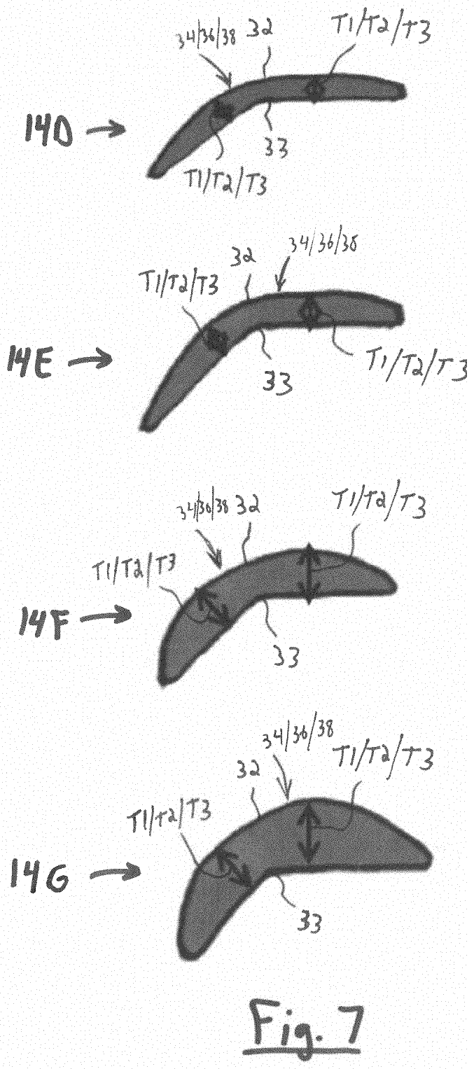

Knee implant systems, kits and/or methods according to the present disclosure may thereby include or utilize a plurality of trochlea components 14, each with respective trochlear grooves 34 configured at differing trochlear angles α (or sulcus orientations). For example, as shown in FIG. 2, a first trochlea component 14A may include a neutrally oriented trochlear groove 34 that is aligned with the orthogonal reference line or plane 27 or includes/defines a trochlear angle α less than or equal to about 2 degrees varus or valgus (i.e., angled from the orthogonal reference line or plane 27 toward the medial or lateral directions when viewed from an anterior vantage point, respectively). As another example, as shown in FIG. 3, a second trochlea component 14B may include a varus oriented trochlear groove 34 that includes/defines a trochlear angle α greater than about 2 degrees varus (i.e., angled from the orthogonal reference line or plane 27 toward the medial direction when viewed from an anterior vantage point, as illustrated in FIG. 3). In some such embodiments, such a trochlea component 14B may include a varus oriented trochlear groove 34 that includes/defines a trochlear angle α within the range of about 2 degrees and 10 degrees varus, or such as within the range of about 2 degrees and 6 degrees varus (e.g., 5 degrees varus). In the exemplary embodiment illustrate in FIG. 3, the trochlea component 14B includes a varus oriented trochlear groove 34 that includes/defines a trochlear angle α of about 4 degrees varus. As yet another example, as shown in FIG. 4, a third trochlea component 14C may include a valgus oriented trochlear groove 34 that includes/defines a trochlear angle α greater than about 2 degrees valgus (i.e., angled from the orthogonal reference line or plane 27 toward the medial direction when viewed from an anterior vantage point, as illustrated in FIG. 4). In some such embodiments, such a trochlea component 14C may include a valgus oriented trochlear groove 34 that includes/defines a trochlear angle α within the range of about 2 degrees and 12 degrees valgus, or such as within the range of about 4 degrees and 8 degrees valgus (e.g., 5 degrees valgus). In the exemplary embodiment illustrate in FIG. 4, the trochlea component 14C includes a valgus oriented trochlear groove 34 that includes/defines a trochlear angle α of about 6 degrees valgus. In some embodiments, femoral implants 10, knee implant systems, kits and/or orthopedic knee surgical procedures of the present discourse may include at least one trochlea component 14 with a neutral trochlear groove 34, at least one trochlea component 14 with a varus oriented trochlear groove 34, at least one trochlea component 14 with a valgus oriented trochlear groove 34, or combinations thereof.

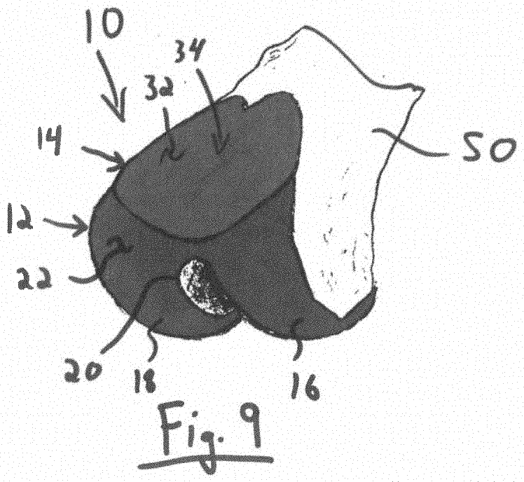

Knee implant systems, kits and/or methods according to the present disclosure may include or utilize a plurality of trochlea components 14 with differing configurations, such as differing configurations of the articular surface 32. A trochlea component 14 that substantially or most closely mimics or recreates the natural, native or constitutional patient's trochlea anatomy, for example, may thereby be selected and utilized for a patient. For example, as shown in FIGS. 6 and 7, the modular trochlea component 14 may be configured or provided with differing configurations of the radius or height/thickness of portions of the articular surface 32, such as differing radiuses or heights/thicknesses of the articular surface 32 at the medial facet portion 36, lateral facet portion 38, trochlear groove portion 34, or a combination thereof.

As shown in FIG. 5, the radius or thickness of a portion of an articular surface of a portion of a femoral implant may be measured or defined with respect to a patellar flexion axis 40. As used herein, a patellar flexion axis 40 of a distal femur is an axis that extends parallel to the cylindrical or transcondylar axis or joint line of the distal femur and passes through the center of a circle 42 that best fits or approximates the radius of the native trochlear groove of the distal femur 50, as shown in FIG. 5.

FIG. 5 also illustrates a typical monoblock femoral implant that is implanted in a conventional way into the distal femur 50 of a patient. As shown, the typical monoblock femoral implant includes at least a trochlea groove that does not anatomically restore the native trochlear groove of the distal femur 50. Rather, as shown by circle 44 that best fits or approximates the radius of the trochlear groove of the typical monoblock femoral implant, the trochlear portion of the implant understuffs the patella-femoral joint (or patellofemoral compartment) such that the groove is positioned closer toward the patellar flexion axis 40 than the native groove/articular surface and/or does not fill the entire length of the native groove. It is noted that other trochlea portions of the typical monoblock femoral implant (e.g., the medial and/or lateral facets) may understuff the patella-femoral joint such that the articular surface portions thereof are positioned closer toward the patellar flexion axis 40 than that of the corresponding native portions and/or do not fill the entire length of the corresponding native portions. It is also noted that some other typical monoblock femoral implants overstuff the patella-femoral joint such that the trochlea groove (and/or the medial and/or lateral facets) is positioned closer toward the patellar flexion axis 40 than the native groove (or other corresponding native surface portion) and/or does not fill the entire length of the native groove (or other corresponding native surface portion). Typical monoblock femoral implants thereby fail to restore the native anatomical patella-femoral joint of patients. The Applicant submits that femoral implants that understuff or overstuff patella-femoral joints result in issues (e.g., instability or immobilization, respectively) during extension and/or flexion, and/or excessively wear the implant.

As shown in FIG. 6, the modular trochlea components 14 (and potentially the condylar component 12) of the femoral implants 10, systems, kits and methods of the present disclosure may include a plurality of differing heights and/or radiuses of the articular surfaces thereof so that the native anatomical patella-femoral joint of a patient can be restored or replicated thereby. For example, in some embodiments a trochlear groove 34 of a first trochlea component may include a first height (or is defined by a first radius) H1 of less than about 19 mm from about 0 degrees to about 100 degrees from the patellar flexion axis 40, the lateral facet portion 38 thereof may include a second height (or is defined by a second radius) H2 of less than about 23 mm from about 0 degrees to about 100 degrees from the patellar flexion axis 40, and/or the medial facet portion 36 thereof includes a second third height (or is defined by a third radius) H3 of less than about 22 mm from about 20 degrees to about 70 degrees from the patellar flexion axis 40, as shown in FIG. 6. As another example, a trochlear groove 34 of a second trochlea component 14 may include a first height (or is defined by a first radius) H1 of about 19 mm from about 0 degrees to about 100 degrees from the patellar flexion axis 40, the lateral facet portion 38 thereof may include a second height (or is defined by a second radius) H2 of about 23 mm from about 0 degrees to about 100 degrees from the patellar flexion axis 40, and/or the medial facet portion 36 thereof includes a second third height (or is defined by a third radius) H3 of about 22 mm from about 20 degrees to about 70 degrees from the patellar flexion axis 40. As yet another example, a trochlear groove 34 of a third trochlea component 14 may include a first height (or is defined by a first radius) H1 of about 21 mm from about 0 degrees to about 100 degrees from the patellar flexion axis 40, the lateral facet portion 38 thereof may include a second height (or is defined by a second radius) H2 of about 26 mm from about 0 degrees to about 100 degrees from the patellar flexion axis 40, and/or the medial facet portion 36 thereof includes a second third height (or is defined by a third radius) H3 of about 25 mm from about 20 degrees to about 70 degrees from the patellar flexion axis 40. In another example, a trochlear groove 34 of a fourth trochlea component 14 may include a first height (or is defined by a first radius) H1 of about 23 mm from about 0 degrees to about 100 degrees from the patellar flexion axis 40, the lateral facet portion 38 thereof may include a second height (or is defined by a second radius) H2 of about 28 mm from about 0 degrees to about 100 degrees from the patellar flexion axis 40, and/or the medial facet portion 36 thereof includes a second third height (or is defined by a third radius) H3 of about 27 mm from about 20 degrees to about 70 degrees from the patellar flexion axis 40. In some embodiments, the first height or radius H1 of a trochlear groove 34 and/or the second height or radius H2 of a lateral facet portion 38 may be a single height or radius. In some embodiments, the third height or radius H3 of a medial facet portion 36 may include a plurality of heights or radiuses.

The modular trochlea components 14 (and potentially the condylar component 12) of the femoral implants 10, systems, kits and methods of the present disclosure may also include a plurality of differing sizes and/or thicknesses so as to restore the native anatomical joint. A trochlea component 14 that substantially or most closely mimics or recreates the natural or native patient's trochlea anatomy, for example, may thereby be utilized. For example, as shown in FIG. 7, modular trochlea components 14 may be configured or provided with differing thicknesses at the medial facet portion 36, lateral facet portion 38, trochlear groove portion 34, or a combination thereof. For example, the maximum thicknesses of the medial facet portion 36, the lateral facet portion 38, and/or the trochlear groove portion 34 measured between the articular surface 32 thereof and an associated engagement surface portion 33 may vary between differing trochlea components 14 (and thereby differing implants 10). The maximum thicknesses of the medial facet portion 36, the lateral facet portion 38, and the trochlear groove portion 34 of a trochlea component 14 may be measured between the articular surface 32 thereof and the associated engagement surface portion 33 along a direction extending normal to the associated engagement surface portion 33 (and/or along a radius defining the articular surface 32 thereof). The tissue engagement surface portions 33 associated with the medial facet portion 36, the lateral facet portion 38, and the trochlear groove portion 34 may be the surface portion that substantially opposes the articular surface 32 and engages the patient's distal femur and/or associated tissue. It is noted that the thickness of the trochlea component 14 (and potentially condylar component 12) may vary along the component, and therefore only one or more portion of the component may be of the maximum thickness and one or more other portion thereof may be thinner than the maximum thickness. In some embodiments, a plurality of modular trochlea components 14 (and potentially the condylar component 12) of a femoral implant 10, system, kit and methods of the present disclosure may include a thinnest trochlea component 14 (and potentially condylar component 12) that defines a minimum thickness of 3 mm or greater, and a plurality of thicker trochlea components 14 (and potentially condylar components 12) with minimum thicknesses that increase incrementally within the range of 2 mm to 5 mm, or 2 mm to 3 mm, from the thinnest component.

For example, as a shown in FIG. 7, the trochlear groove 34 of a first trochlea component 14D may include a maximum thickness T1 such that the lateral facet height H1 thereof (see FIG. 6) is less than about 19 mm from about 0 degrees to about 100 degrees from the patellar flexion axis 40, as discussed above. As also shown in FIG. 7, the lateral facet portion 38 of the first trochlea component 14D may include a maximum thickness T2 such that the lateral facet height H2 thereof (see FIG. 6) is less than about 23 mm from about 0 degrees to about 100 degrees from the patellar flexion axis 40, as discussed above. As further shown in FIG. 7, the medial facet portion 36 of the first trochlea component 14D may include a maximum thickness T3 such that the medial facet height H3 thereof (see FIG. 6) is less than about 22 mm within the range about 20 degrees to about 70 degrees of revolving process from the patellar flexion axis 40, as discussed above.

As another example, as shown in FIG. 7, a second trochlea component 14E may define a maximum thickness in at least one portion that is larger than that of the first trochlea component 14D. As a shown in FIG. 7, the c of a second trochlea component 14E may include a maximum thickness T1 such that the lateral facet height H1 thereof (see FIG. 6) is about 19 mm from about 0 degrees to about 100 degrees from the patellar flexion axis 40, as discussed above. As also shown in FIG. 7, the lateral facet portion 38 of the second trochlea component 14E may include a maximum thickness T2 such that the lateral facet height H2 thereof (see FIG. 6) is about 23 mm from about 0 degrees to about 100 degrees from the patellar flexion axis 40, as discussed above. As further shown in FIG. 7, the medial facet portion 36 of the second trochlea component 14E may include a maximum thickness T3 such that the medial facet height H3 thereof (see FIG. 6) is about 22 mm within the range about 20 degrees to about 70 degrees of revolving process from the patellar flexion axis 40, as discussed above.

As another example, as shown in FIG. 7, a third trochlea component 14F may define a maximum thickness in at least one portion that is larger than that of the second trochlea component 14D. As a shown in FIG. 7, the trochlear groove 34 of a third trochlea component 14F may include a maximum thickness T1 that is at least about 2 mm greater than the maximum thickness T1 of the trochlear groove 34 of the second trochlea component 14E. As also shown in FIG. 7, the lateral facet portion 38 of the third trochlea component 14F may include a maximum thickness T2 that is at least about 3 mm greater than the maximum thickness T2 of the lateral facet portion 38 of the second trochlea component 14E. As further shown in FIG. 7, the medial facet portion 36 of the third trochlea component 14F may include a maximum thickness T3 that is at least about 3 mm greater than the maximum thickness T2 of the lateral facet portion 38 of the second trochlea component 14E. Similarly, as another example shown in FIG. 7, a fourth trochlea component 14G may define a maximum thickness in at least one portion that is larger than that of the third trochlea component 14E. As a shown in FIG. 7, the trochlear groove 34 of the third trochlea component 14F may include a maximum thickness T1 that is at least about 4 mm greater than the maximum thickness T1 of the trochlear groove 34 of the second trochlea component 14E. As also shown in FIG. 7, the lateral facet portion 38 of the fourth trochlea component 14G may include a maximum thickness T2 that is at least about 5 mm greater than the maximum thickness T2 of the lateral facet portion 38 of the second trochlea component 14E. As further shown in FIG. 7, the medial facet portion 36 of the fourth trochlea component 14G may include a maximum thickness T3 that is at least about 5 mm greater than the maximum thickness T2 of the lateral facet portion 38 of the second trochlea component 14E.

In this way, the present disclosure provides for femoral implants 10, systems, kits and methods that include a plurality of modular trochlea components 14 that differ with respect to at least one of the orientation/alignment of the trochlea groove 34 thereof (e.g., varus, neutral or valgus), the height or radius of the trochlea groove 34, medial facet portion 36 and/or lateral facet 38 of the articular surface thereof, the thickness thereof (e.g., minimum thickness, maximum thickness, or thickness profile (i.e., variance in thickness)) (e.g., thicknesses incrementally increasing by 2 or 3 mm), or combinations thereof. One particular modular trochlea component 14 of the plurality of differing modular trochlea components 14 (and potentially a may thereby be selected for a particular patient to cooperate with a condylar component 12 (e.g., any of a plurality of differing condylar components 12, if provided) to form a femoral implant 10 that mimics or recreates patient's natural or native anatomy (and thereby the patient's native tibio-femoral joint), such as the patient's native trochlea anatomy (and thereby the patient's native patello-femoral joint).

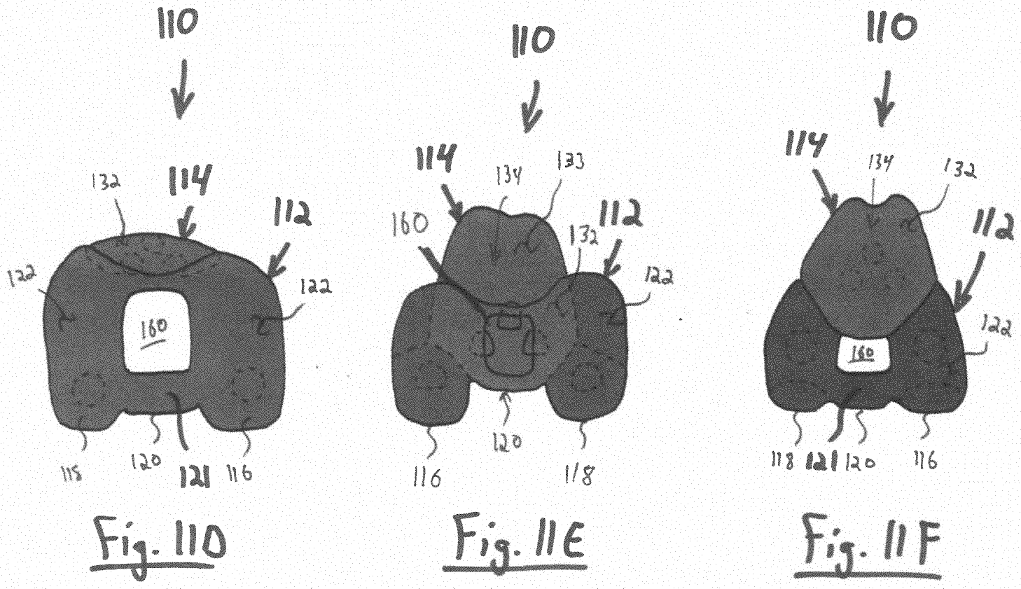

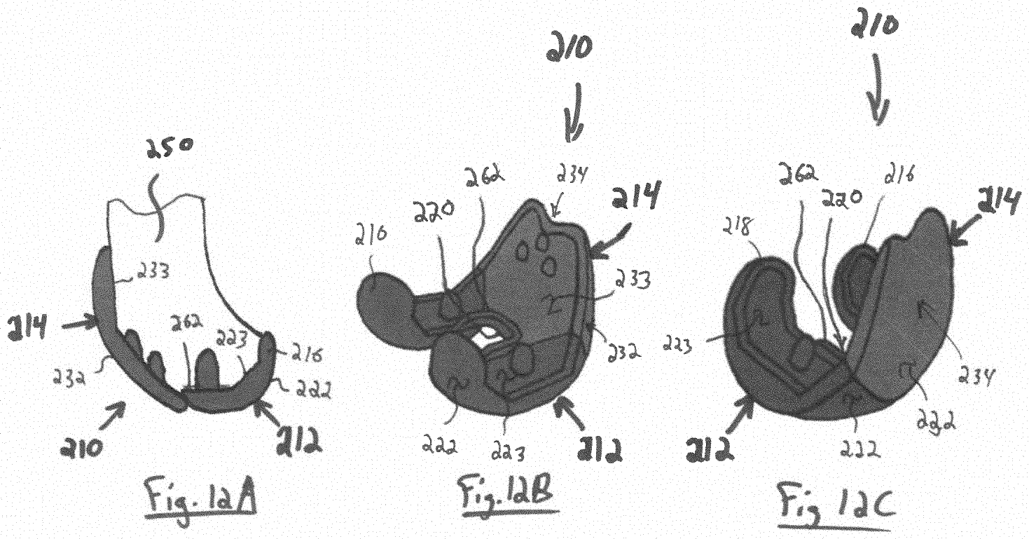

As shown in FIGS. 9 and 10, the engagement side or surface 33 of the trochlea component 14 and an engagement side or surface 23 of the condylar component 12 (which may substantially oppose the articular surface 32 thereof) may contact or engage a surgically-prepared distal femur 50 of a patient. The engagement sides 23, 33 of the femoral implant 10 may include one or more surfaces that mate with one or more surfaces 52 that are surgically prepared or cut into the patient's distal femur 50, such as planar surfaces as shown in FIG. 8. As also shown in FIG. 1, the engagement side 23 of the condylar component 12 and/or the engagement side 33 of the trochlear component 14 may include one or more pegs or projections 72 extending therefrom. For example, as shown in FIG. 1, in some embodiments the condylar component 12 may include a peg 72 projecting from the engagement surface 23 of the medial condyle portion 116 and a peg 72 projecting from the engagement surface 23 of the lateral condyle portion 118. As also shown in FIG. 1, in some embodiments the trochlea component 14 may include a plurality of pegs 72 projecting from the engagement surface 33. As shown in FIG. 1, in some embodiments, the trochlea component may include a first medially-positioned peg 72, a second laterally-positioned peg 72, and a third peg positioned at least partially between the first and second pegs 72 along the medial-lateral direction. The first and second pegs 72, 72 may be aligned along the distal-proximal direction, and/or the third peg 72 may be positioned proximally of the first and second pegs 72, 72, as shown in FIG. 1. In some embodiments, the trochlea component 14 may include a fourth peg (not shown) that is positioned between the first and second pegs 72 along the medial-lateral direction and/or distally from the first and second pegs 72, 72.

A method of substantially replicating at least a portion of a native articular surface portion of the distal femur 50 may include preparing the distal femur 50, at least in part, for implantation of the femoral implant 10. In some embodiments, the orientation (e.g., varus/valgus orientation) of the native trochlea groove of the distal femur 50 may be marked or otherwise identified on the distal femur 50. For example, at least a pair of cavities (e.g., via a drill) or marks (e.g., via a surgical pen) may be made into/on the distal femur 50 at/in the native trochlea groove to indicate its varus/valgus orientation. In some embodiments, the position (e.g., medial-lateral position) of the native trochlea groove 34 of the distal femur 50 may also be marked or otherwise identified on the distal femur 50. For example, a cavity (e.g., via a drill) or mark (e.g., via a surgical pen) may be made into/on the distal femur 50 at/in a distal portion of the native trochlea groove 34 to indicate the medial-lateral position thereof.

The method of substantially replicating at least a portion of a native articular surface portion of the distal femur 50 may include cutting or otherwise removing a portion of the distal femur 50 to form a prepared bone surface 52 configured to engage with the engagement side 23 of the condylar component 12 and/or the engagement side 33 of the trochlear component 14. For example, anterior and/or posterior chamfered surfaces 52 may be formed on the distal femur 50, as shown in FIG. 8. In some embodiments, at least a portion of the prepared bone surface 52 may be formed prior to the formation of cavities 54, 56 in the distal femur 50 (as described further below) and/or at least a portion of the prepared bone surface 52 may be formed subsequent to the formation of the cavities 54, 56 in the distal femur 50. For example, in one example, at least an anterior femoral cut may be made in the distal femur 50 to form an anterior prepared surface portion of the prepared bone surface 52 prior to the formation of cavities 54, 56 in the distal femur 50.

In some embodiments, after at least partially preparing the distal femur 50 by forming the prepared bone surface 52, an orientation line between the cavities or marks into/on the distal femur 50 that indicate the orientation (e.g., varus/valgus orientation) of the native trochlea groove may be formed. The orientation line may thereby indicate the orientation (e.g., varus/valgus orientation) of the native trochlea groove 34. In some embodiments, the orientation line may be formed into/on the prepared bone surface 52 (e.g., via surgical marker).

As shown in FIG. 8, the method of substantially replicating at least a portion of a native articular surface portion of the distal femur 50 may further include preparing the distal femur 50 via a guide 70. The guide 70 may be configured to facilitate forming one or more condylar cavities or apertures 54 in the condylar portion of the distal femur 50 and/or one or more trochlear cavities or apertures 56 in the trochlear portion of the distal femur 50 to accommodate or mate with the pegs 72 of the engagement sides 23, 33 of the femoral implant 10, as shown in FIG. 8. For example, the guide 70 may include at least one condylar aperture 74 configured to facilitate the formation of the one or more condylar cavities 54 within the distal femur 50 and/or at least one trochlear aperture 76 configured to facilitate the formation of the one or more trochlear cavities 56 within the distal femur 50, as shown in FIG. 8. The guide 70 may be configure as a drill guide such that the at least one condylar aperture 74 and the at least one trochlear aperture 76 can be utilized by a drill to form the one or more condylar cavities 54 and one or more trochlear cavities 56, respectively, in the distal femur 50. The guide 70 may be positioned medial-laterally on the prepared bone surface 52 of the distal femur 50 in a position that matches or corresponds to the medio-lateral position of the prosthetic and the native distal trochlea groove 34.

In some embodiments, a knee or femoral prosthesis kit of the present disclosure may include at least one condylar component 12, a plurality of modular trochlea components 14 and the guide 70. As discussed above, the plurality of modular trochlea components 14 of the system and kit may include differing trochlea groove orientations, thicknesses and/or articular surface 32 radiuses/shape, as well as trochlea component sizes (e.g., differing medial-lateral widths, proximal-distal lengths and/or anterior-posterior depths). For example, an optimal modular trochlea component 14 may be chosen from the plurality of differing modular trochlea components 14 by matching the orientation of a modular trochlea component 14 with the native trochlea groove orientation indicated by the at least a pair of cavities and/or the orientation line. An optimal modular trochlea component 14 may also be chosen from the plurality of differing modular trochlea components 14 by matching the trochlea thickness of a modular trochlea component 14 with the thickness between the native trochlea groove orientation and the prepared bone surface 52 (e.g., which may be determined by the thickness of the removed trochlea bone and in consideration of cartilage wear and a kerf of the saw blade, for example). An optimal modular trochlea component 14 may also be chosen from the plurality of differing modular trochlea components 14 by matching the medial-distal width, proximal-distal length and/or anterior-posterior depth of a modular trochlea component 14 with that of the native distal femur 50.

In some embodiments, the method of substantially replicating at least a portion of a native articular surface portion of the distal femur 50 may include resurfacing the patella of the patient proximate to the distal femur 50. In such embodiments, two or more cavities (e.g., via a drill) or other marks may be made along the native crest before the patella is cut or otherwise resurfaced. Subsequent to the resurfacing of the patella, a line may be made (e.g., via a surgical marker) between the two or more cavities or other marks, and the medial-lateral position and frontal position of the crest of the optimal modular trochlea component 14 and the crest of the native distal femur 50 may be matched or made consistent (via the line).

The engagement side 23 of the condylar component 32 and/or the engagement side 33 of the trochlear component 14 may be fixedly coupled to the surgically-prepared surface 52 of the distal femur 50 via use of bone cement and/or any other attachment mechanism. As noted above and shown in FIGS. 9 and 10, the condylar component 12 and the trochlea component 14 may be coupled to the surgically-prepared surface 52 of a distal femur 50 in a cooperating or coupled arrangement to form at least portion of the distal aspect of the femur, as shown in FIGS. 9 and 10. The articular surface 32 of the trochlea component 14 and the articular surface 22 of the condyle component 12 may thereby cooperate to form a substantially continuous or uniform outer articulation surface of the femoral implant 10, as shown in FIGS. 9 and 10 (e.g., extending from a joint therebetween). As shown in FIG. 10, in some embodiments the trochlea component 14 and the condyle component 12 may overlap at the joint therebetween. For example, a relatively thin portion of the trochlea component 14 may overlap an underlying relatively thin portion of the condyle component 12 (or vice versa), and such relatively thin portions may be coupled to each other via cement or another adhesive for example. The engagement surfaces of 23, 33 of the condyle component 12 and the trochlea component 14 may also be coupled to the surgically-prepared surface 52 of a distal femur 50 as shown in FIG. 10, such as via cement, another adhesive and/or a mechanical interlocking mechanism for example.

In this way, the present disclosure provides for femoral implants 10, systems, kits and methods that include a plurality of modular trochlea components 14 that differ with respect to at least one of the orientation/alignment of the trochlea groove 34 thereof (e.g., varus, neutral or valgus), the height or radius of the trochlea groove 34, medial facet portion 36 and/or lateral facet 38 of the articular surface thereof, the thickness thereof (e.g., minimum thickness, maximum thickness, or thickness profile (i.e., variance in thickness)) (e.g., thicknesses incrementally increasing by 2 or 3 mm), or combinations thereof.

The implants 10, systems, kits and related methods may thereby include modular trochlea components 14 (and potentially a plurality of trochlea components 12) in multiple sizes (e.g., size 1, size 2, size 3, etc.) that incrementally increase in medial-distal width, proximal-distal length and/or anterior-posterior depth. The implants 10, systems, kits and related methods may thereby include modular trochlea components 14 would thereby also include a plurality of differing modular trochlea components 14 (and potentially a plurality of trochlea components 12) in each size thereof. For example, each size of the plurality of modular trochlea components 14 may include a plurality of modular trochlea components 14 that differ in the orientations of the trochlea groove 34 (e.g., varus/valgus orientations, as discussed above), thicknesses (e.g., trochlea groove 34 thicknesses) and/or articular surface 32 radiuses/shapes. The plurality of modular trochlea components 14 (and potentially the plurality of trochlea components 12) of the differing sizes, and/or the differing modular trochlea components 14 (and potentially the plurality of trochlea components 12) of each differing size, may include the same relative locations or positions of the pegs 72. In use, even after the cavities 56, 54 are formed into the distal femur 50, the surgeon can select an optimal modular trochlea component 14 from the plurality of differing modular trochlea components 14 (and potentially an optimal trochlea components 12 of a plurality of differing trochlea components 12) that best replicates at least a portion of the native articular surface of the distal femur 50 without having to adjust or re-form the cavities 56, 54. In this way, one particular modular trochlea component 14 of the plurality of differing modular trochlea components 14 may be selected for a particular patient to cooperate with a condylar component 12 (e.g., any of a plurality of differing condylar components 12, if provided) to form a femoral implant 10 that mimics or recreates the patient's natural or native distal femoral anatomy (and thereby the patient's native tibio-femoral joint), such as the patient's native trochlea anatomy (and thereby the patient's native patello-femoral joint).

As noted above, a type of knee prosthesis used to replace a patient's natural knee may also depend on whether the patient's posterior cruciate ligament is retained or sacrificed (i.e., removed) during surgery. In some embodiments, if the patient's posterior cruciate ligament is damaged, diseased and/or otherwise removed, a posterior stabilized knee implant or prosthesis may be used to provide additional support and/or control at later degrees of flexion. An exemplary posterior stabilized femoral prosthesis 110 is shown in FIGS. 11A-11F. Exemplary femoral implant 110 is substantially similar to exemplary femoral implant 10 described above in connection with FIGS. 1A-10, and therefore like reference numerals preceded by the number “1” are used to indicate like elements or features. The description above with reference to femoral implant 10 may therefore equally apply to the particular components, features or the like of exemplary femoral implant 110 and is not repeated hereinafter for brevity sake.

As shown in FIGS. 11A-11F, the condylar component 112 of the femoral implant 110 differs from the condylar component 12 of the femoral implant 10. As shown in FIGS. 11B-11F, the condylar component 112 includes a postero-stabilized design. As shown in FIGS. 11B-11F, the condylar component 112 includes a cross or tie portion or member 121 that extends between the medial and lateral condyle portions 116, 118. In some embodiments, the cross portion 121 may form at least part of the intermediate articular surface portion of the articular surface 122 of the condylar component 112. However, the trochlea groove 134 of the implant 110 may only extend along (or be formed by) the trochlea component 114.

The cross portion 121 may form the intercondylar notch 120 further posteriorly and proximately as compared to the intercondylar notch 20 of the condylar component 12 discussed above, as shown in FIGS. 11B-11D and 11F. As shown in FIGS. 11B-11F, the cross portion 121 may partially form an aperture 160 that extends through the distal portion of the component 112. The aperture 160 may be positioned between the medial and lateral articular surface portions of the articular surface 122 along the medial-lateral direction and anteriorly of the cross portion 121 of (and thereby the intercondylar notch 120).

As shown in FIGS. 11B-11, the aperture 160 may be configured to accept a cam or post of a bearing surface or implant (not shown) to extend therein or therethrough. The cam or post and the side walls of the aperture 160 of the condylar component 112 may thereby prevent translation or movement of the condylar component 112 (and thereby the femur that the condylar component 112 is coupled to) and the bearing surface or implant (and thereby the tibia that the bearing surface or implant is coupled to).

As shown in FIGS. 11A-11C, the condylar component 112 of the femoral implant 110 includes a wall or projection 162 extending from the engagement surface 123 proximate to the aperture 160. In some embodiments, the projection 162 extends about the periphery of the aperture 160, as shown in FIGS. 11A-11C. The peripheral projection 162 may be configured to extend into the intercondylar area of a distal femur (typically occupied by the anterior and/or posterior cruciate ligaments attaching to the femur). The aperture 160 and the projection 162 may thereby form an intercondylar box that accepts a stabilizing post or cam therein to stabilize the femoral knee implant 110 and a bearing surface or implant (and/or the knee joint as a whole) through a patient's range of motion.

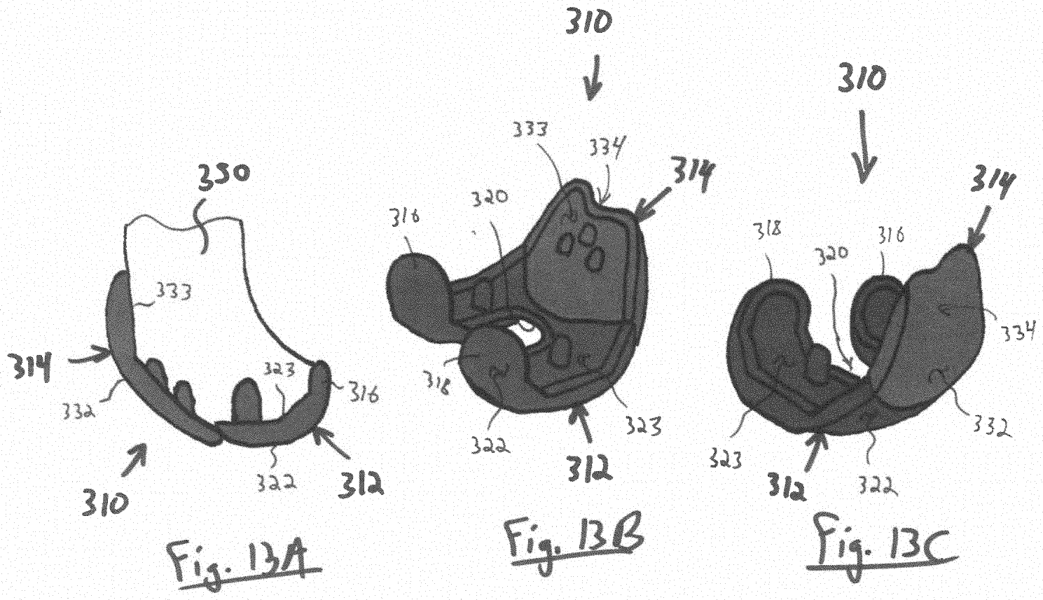

As noted above, in some embodiments a patient's posterior cruciate ligament is retained, and therefore a femoral implant may be configured to accommodate the posterior cruciate ligament. An exemplary cruciate and/or bi-cruciate retaining femoral prosthesis 210 is shown in FIGS. 12A-12F. Exemplary femoral implant 210 is substantially similar to exemplary knee implant 110 described above in connection with FIGS. 11A-11F, and therefore like reference numerals preceded by the number “2” as opposed to “1” are used to indicate like elements or features. The description above with reference to femoral implant 110 may therefore equally apply to the particular components, features or the like of exemplary femoral implant 210 and is not repeated hereinafter for brevity sake.

As shown in FIGS. 12B-12F, the condylar component 212 of the femoral implant 210 is void of the cross portion 121 as described above with respect to condylar component 112 of the femoral implant 110 of FIGS. 11A-11F. As such, the intercondylar notch 220 extends anteriorly proximate to the trochlea component 114, as shown in FIGS. 12B-12F. As shown in FIGS. 12B and 12C, the projection 262 at the engagement surface 223 extends partially along the periphery of the intercondylar notch 220. For example, the projection 262 may extend along the anterior side of the intercondylar notch 220 and/or partially along the interior sides of the medial and lateral condyle portions 116, 118, as shown in FIGS. 12B and 12C. The condylar component 212 may thereby extend into the intercondylar area of a distal femur while allowing the anterior and/or posterior cruciate ligaments to also extend through the intercondylar area.

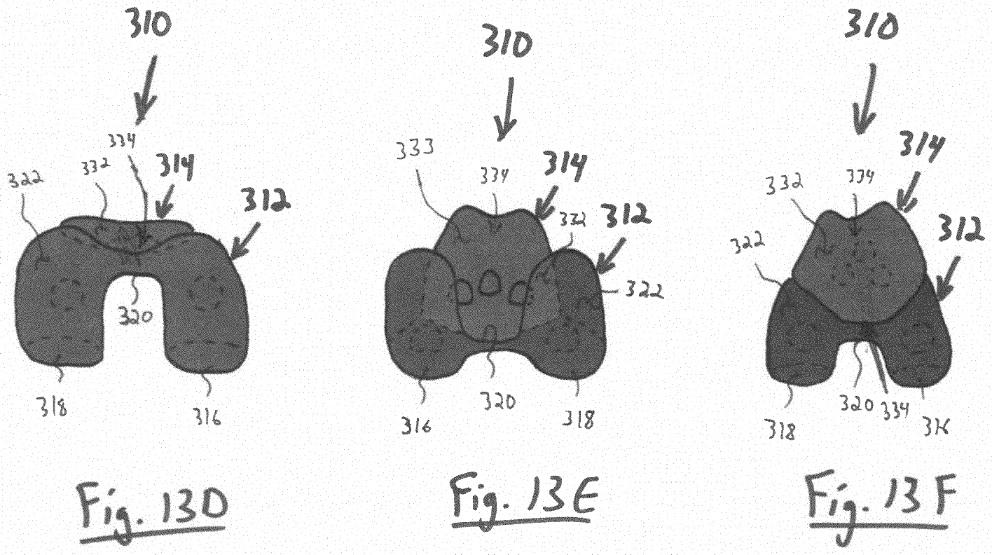

Another exemplary cruciate and/or bi-cruciate retaining femoral prosthesis 310 is shown in FIGS. 13A-13F. Exemplary femoral implant 310 is substantially similar to exemplary knee implant 210 described above in connection with FIGS. 12A-12F, and therefore like reference numerals preceded by the number “3” as opposed to “2” are used to indicate like elements or features. The description above with reference to femoral implant 210 may therefore equally apply to the particular components, features or the like of exemplary femoral implant 310 and is not repeated hereinafter for brevity sake.

As shown in FIGS. 13B and 13C, exemplary femoral implant 310 differs from exemplary femoral implant 210 with respect to the engagement side 323 of the condylar component 312. The engagement side 323 of the condylar component 312 is void of a projection extending along a portion of the periphery of the intercondylar notch 320, as shown in FIGS. 13B and 13C. The condylar component 312 may thereby not extend into an intercondylar area of a distal femur when coupled thereto. Further, the intercondylar notch 320 may allow the anterior and/or posterior cruciate ligaments of the patient's knee to extend through the intercondylar area.

The exemplary femoral implant 310 also differs from exemplary femoral implant 210 with respect to the articular surface 322 of the condylar component 312. As shown in FIGS. 13D and 13F, both the articular surface 332 of the trochlea component 314 and the articular surface 322 of the condylar component 312 define the trochlea groove 334. For example, the intermediate articular surface portion of the articular surface 322 of the condylar component 312 may define a distal portion of the trochlea groove 334, as shown in FIGS. 13D and 13F.

The terminology used herein is for the purpose of describing particular embodiments only and is not intended to be limiting of an invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), “contain” (and any form contain, such as “contains” and “containing”), and any other grammatical variant thereof, are open-ended linking verbs. As a result, a method or article that “comprises”, “has”, “includes” or “contains” one or more steps or elements possesses those one or more steps or elements, but is not limited to possessing only those one or more steps or elements. Likewise, a step of a method or an element of an article that “comprises”, “has”, “includes” or “contains” one or more features possesses those one or more features, but is not limited to possessing only those one or more features.

As used herein, the terms “comprising,” “has,” “including,” “containing,” and other grammatical variants thereof encompass the terms “consisting of” and “consisting essentially of.”

The phrase “consisting essentially of” or grammatical variants thereof when/if used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed compositions or methods.

Any publication cited in this specification are herein incorporated by reference as if each individual publication were specifically and individually indicated to be incorporated by reference herein as though fully set forth. Any subject matter incorporated by reference is not considered to be an alternative to any claim limitations, unless otherwise explicitly indicated.

Where one or more ranges are referred to throughout this specification, each range is intended to be a shorthand format for presenting information, where the range is understood to encompass each discrete point within the range as if the same were fully set forth herein.

While several aspects and embodiments of the present disclosure have been described and depicted herein, alternative aspects and embodiments may be affected by those skilled in the art to accomplish the same objectives. Accordingly, this disclosure and the appended claims are intended to cover all such further and alternative aspects and embodiments as fall within the true spirit and scope of inventions of the present disclosure.

I claim:

1. A femoral implant system for a knee replacement prosthesis, comprising:
at least one condylar component comprising a medial condyle portion defining a medial condyle articular surface portion, a lateral condyle portion defining a lateral condyle articular surface portion, and an intercondylar notch formed between the medial and lateral condyles; and
a plurality of modular trochlea components each comprising an articular surface that defines a trochlea groove, the trochlea grooves of the plurality of modular trochlea components differing from each other,
wherein the at least one condylar component and the plurality of modular trochlea components are separate components, and
wherein one condylar component of the at least one condylar component and one modular trochlea component of the plurality of modular trochlea components are configured to be implanted into a distal end portion of a femur and cooperate to form replacement articulation surfaces that substantially replicate at least a portion of a native articular surface portion of the distal end portion of the femur.

2. The system of claim 1, wherein the plurality of modular trochlea components comprises a plurality of modular trochlea components with differing orientations of the trochlea grooves thereof.

3. The system of claim 2, wherein the plurality of modular trochlea components comprises a plurality of modular trochlea components with differing maximum thicknesses between the articular surfaces and engagement surfaces thereof.

4. The system of claim 3, wherein the plurality of modular trochlea components comprises a plurality of modular trochlea components with differing medial-lateral widths and proximal-distal lengths.

5. The system of claim 2, wherein the plurality of modular trochlea components comprises at least one modular trochlea component with a neutrally oriented trochlea groove that is angled less than or equal to about 1 degree from an axis that is normal to a distal femoral joint line defined by the medial and lateral condyles of the one condylar component.

6. The system of claim 2, wherein the plurality of modular trochlea components comprises at least one modular trochlea component with a varus oriented trochlea groove that is angled greater than about 1 degree in the medial direction from an axis that is normal to a distal femoral joint line defined by the medial and lateral condyles of the one condylar component.

7. The system of claim 2, wherein the plurality of modular trochlea components comprises at least one modular trochlea component with a valgus oriented trochlea groove that is angled greater than about 1 degree in the lateral direction from an axis that is normal to a distal femoral joint line defined by the medial and lateral condyles of the one condylar component.

8. The system of claim 2, wherein the plurality of modular trochlea components comprises:
at least one modular trochlea component with a neutrally oriented trochlea groove that is angled less than or equal to about 1 degree from an axis that is normal to a distal femoral joint line defined by the medial and lateral condyles of the one condylar component;
at least one modular trochlea component with a varus oriented trochlea groove that is angled greater than about 1 degree in the medial direction from an axis that is normal to a distal femoral joint line defined by the medial and lateral condyles of the one condylar component; and
at least one modular trochlea component with a valgus oriented trochlea groove that is angled greater than about 1 degree in the lateral direction from an axis that is normal to a distal femoral joint line defined by the medial and lateral condyles of the one condylar component.

9. The system of claim 2, wherein the trochlea groove of the one modular trochlea component of the plurality of modular trochlea components substantially corresponds to the orientation of a native anatomic trochlea groove of the distal end of the femur.

10. The system of claim 1, wherein the trochlea grooves of the plurality of modular trochlea components are each defined by at least one radius of curvature, and wherein the plurality of modular trochlea components comprises a plurality of modular trochlea components with differing radiuses of curvature.

11. The system of claim 10, wherein the plurality of modular trochlea components comprises modular trochlea components with differing maximum thicknesses between the trochlea grooves and engagement surfaces thereof.

12. The system of claim 1, wherein the at least one condylar component comprises an intermediate articular surface portion extending medially-laterally between the medial condyle articular surface portion and the lateral condyle articular surface portion, and wherein a distal portion of the intermediate articular surface portion comprises a supplemental trochlea groove portion that cooperates with the trochlea groove of the one modular trochlea component.

13. The system of claim 1, wherein the at least one condylar component comprises an intermediate articular surface portion extending medially-laterally between the medial condyle articular surface portion and the lateral condyle articular surface portion, and wherein the intermediate articular surface portion is void of a groove that cooperates with the trochlea groove of the one modular trochlea component.

14. The system of claim 1, wherein the articular surface of each of the plurality of modular trochlea components defines a medial articular surface portion on a medial side of the trochlea groove and a lateral articular surface portion on a lateral side of the trochlea groove that are convex medially-laterally, and wherein the trochlea groove is concave medially-laterally.

15. The system of claim 1, wherein the at least one condylar component comprises an engagement side with first pegs, and the plurality of modular trochlea components each comprise an engagement side with second pegs, the second pegs of the plurality of modular trochlea components being in the same relative positions.

16. A kit, comprising:
at least one condylar component comprising an engagement side with first pegs, and an articular side with a medial condyle articular surface portion, a lateral condyle articular surface portion, and an intercondylar notch between the medial and lateral condyle articular surface portions;
a plurality of modular trochlea components each comprising an engagement side with second pegs, and an articular side with an articular surface that defines a trochlea groove, the trochlea grooves of the plurality of modular trochlea components differing from each other; and
a guide comprising a plurality of apertures, the guide configured to facilitate the formation of a plurality of cavities within a distal end portion of a femur via the plurality of apertures for mating with the first pegs of one condylar component of the at least one condylar component and with the second pegs of one modular trochlea of the plurality of modular trochlea components,
wherein the at least one condylar component and the plurality of modular trochlea components are separate components, and
wherein the one condylar component and the one modular trochlea component cooperate to form replacement articulation surfaces of the distal end portion of the femur that substantially replicate at least a portion of a native anatomic articular surface portion of the distal end portion of the femur.

17. The kit of claim 16, wherein the second pegs of the plurality of modular trochlea components are in the same positions.

18. The kit of claim 16, wherein the plurality of modular trochlea components comprises a plurality of modular trochlea components with differing orientations of the trochlea groove thereof, a plurality of modular trochlea components with differing maximum thicknesses between the articular surfaces and engagement surfaces thereof, and a plurality of modular trochlea components with differing medial-lateral widths and proximal-distal lengths.

19. A method of substantially replicating at least a portion of a native anatomic articular surface portion of a distal end portion of a femur, comprising:
selecting a first modular trochlea component from a plurality of modular trochlea components with a first trochlea groove that substantially corresponds to a native trochlea groove of the distal end portion of the femur, each of the plurality of modular trochlea components comprising an articular surface that defines a differing trochlea groove;
coupling a condylar component to the distal end portion of the femur, the condylar component comprising a medial condyle portion defining a medial condyle articular surface portion, a lateral condyle portion defining a lateral condyle articular surface portion, and an intercondylar notch formed between the medial and lateral condyles; and
coupling the first modular trochlea component to the distal end portion of the femur such that the first modular trochlea component and the condylar component cooperate to form replacement articulation surfaces of the distal end portion of the femur that substantially replicate at least a portion of the native anatomic articular surface portion of the distal end portion of the femur.

20. The method of claim 19, wherein the plurality of modular trochlea components comprises modular trochlea components with differing orientations of the trochlea groove thereof, and wherein selecting the first modular trochlea component comprises selecting a modular trochlea component from the plurality of modular trochlea components with a trochlea groove that includes an orientation that substantially corresponds to the orientation of the native anatomic trochlea groove of the distal end portion of the femur.
